(19) United States
(12) Patent Application Publication
(10) Pub. No.: US 2024/0094187 A1
(43) Pub. Date: Mar. 21, 2024

(54) SURFACE ENHANCED RAMAN SPECTROSCOPIC METHODS FOR DETECTING ANALYTES

(71) Applicants: Beckman Coulter, Inc., Brea, CA (US); Johns Hopkins University, Baltimore, MD (US)

(72) Inventors: Ishan Barman, Baltimore, MD (US); Peng Zheng, Ellicott City, MD (US); Taka Mizutani, Brea, CA (US)

(73) Assignees: Beckman Coulter, Inc., Brea, CA (US); Johns Hopkins University, Baltimore, MD (US)

(21) Appl. No.: 18/259,727

(22) PCT Filed: Dec. 29, 2021
(86) PCT No.: PCT/US2021/065526

§ 371 Date: Jun. 28, 2023

Related U.S. Application Data
(60) Provisional application No. 63/132,248, filed on Dec. 30, 2020.

(51) Int. Cl. G01N 33/487; G01N 21/65; G01N 33/543

(52) U.S. Cl. CPC G01N 33/487; G01N 21/658; G01N 33/543; B82Y 30/00

(57) ABSTRACT

The disclosure relates to a substrate comprising a micro- or nanostructured periodic array comprised of a plurality of anisotropic metallic micro- or nanostructures, wherein each of the plurality of nanostructures induce an average maximum and substantially uniform plasmonic field greater than 10⁸ across the substrate; a plurality of Raman-active linker molecules directly bound to the metallic micro- or nanostructures; and a plurality of capture molecules directly bound to the Raman-active linker molecules. The disclosure also relates to systems, devices, and methods that use the substrates to determine the concentration of various analytes.

### CROSS-REFERENCE TO RELATED APPLICATIONS

This application claims the benefit of U.S. Provisional Appl. Ser. No. 63/132,248, filed Dec. 30, 2020, which is incorporated by reference as if fully set forth herein.

### BACKGROUND

When a molecule is irradiated with photons of a particular frequency, the photons are scattered. The majority of the incident photons are elastically scattered without a change in frequency (Rayleigh scattering), whereas a fraction of the incident photons interact with a vibrational mode of the irradiated molecule and are inelastically scattered. The inelastically scattered photons are shifted in frequency and have either a higher frequency (anti-Stokes) or a lower frequency (Stokes). By plotting the frequency of the inelastically scattered photons against their intensity, a unique Raman spectrum of the molecule is observed. The low sensitivity of conventional Raman spectroscopy, however, has limited its use for characterizing biological samples in which the target analyte(s) typically are present in small quantities.

When a Raman-active molecule is adsorbed on or in close proximity to, e.g., within about 5 nm, a metal surface, the intensity of a Raman signal arising from the Raman-active molecule can be enhanced. This enhancement is referred to as the surface-enhanced Raman scattering (SERS) effect. The SERS effect was first reported in 1974 by Fleishman et al., who observed intense Raman scattering from pyridine adsorbed on a roughened silver electrode surface. See Fleishman et al., “Raman spectra of pyridine adsorbed at a silver electrode,” Chem. Phys. Lett., 26, 163 (1974); see also Jeanmaire, D. L., and Van Dyne, R. P., “Surface Raman spectroelectrochemistry. 1. Heterocyclic, aromatic, and aliphatic-amines absorbed on anodized silver electrode.” J. Electroanal. Chem., 84(1), 1-20 (1977); Albrecht, M. G., and Creighton, J. A., “Anonymously intense Raman spectra of pyridine at a silver electrode,” J.A.C.S., 99, 5215-5217 (1977). Since then, SERS has been observed for a number of different molecules adsorbed on the surface of metal surfaces. See, e.g., A. Campion, A. and Kambhampati, P., “Surface-enhanced Raman scattering,” Chem. Soc. Rev., 27, 241 (1998).

The magnitude of the SERS enhancement depends on a number of parameters, including the position and orientation of various bonds present in the adsorbed molecule with respect to the electromagnetic field at the metal surface. The mechanism by which SERS occurs is thought to result from a combination of (i) surface plasmon resonances in the metal that enhance the local intensity of the incident light; and (ii) formation and subsequent transitions of charge-transfer complexes between the metal surface and the Raman-active molecule.

The SERS effect can be observed with Raman-active molecules adsorbed on or in close proximity to metal colloidal particles, metal films on dielectric substrates, and metal particle arrays, including metal nanoparticles. For example, Kneipp et al. reported the detection of single molecules of a dye, cresyl violet, adsorbed on aggregated clusters of colloidal silver nanoparticles. See Kneipp, K. et al., “Single molecule detection using surface-enhanced Raman scattering (SERS), Phys. Rev. Lett., 78(9), 1667-1670 (1997). Nie and Emory observed the surfaced enhanced resonance Raman spectroscopy (SERRS) signal, wherein the resonance between the absorption energy of the Raman-active molecule and that of the nanoparticle yield an enhancement as large as about 10¹⁰ to about 10¹², of a dye molecule adsorbed on a single silver nanoparticle, where the nanoparticles ranged from spherical to rod-like and had a dimension of about 100 nm. See Nie, S., and Emory, S. R., “Probing single molecules and single nanoparticles by surface-enhanced Raman scattering,” Science, 275, 1102-1106 (1997); Emory, S. R., and Nie, S., “Near-field surface-enhanced Raman spectroscopy on single silver nanoparticles,” Anal. Chem., 69, 2631 (1997).

Even with the enhanced signal due to the SERS or SERRS effect, the use of Raman spectroscopy can be limited in diagnostic assays and applications requiring a high sensitivity. Accordingly, there is a need in the art for SERS-active reporter molecules that give rise to an increased Raman signal when compared to SERS-active reporter molecules known in the art.

### SUMMARY

The instant disclosure addresses, in whole or in part, the needs in the art.

The disclosure relates to a substrate comprising a micro- or nanostructured periodic array comprised of a plurality of anisotropic metallic micro- or nanostructures, wherein each of the plurality of nanostructures induce an average maximum and substantially uniform plasmonic field greater than 10⁸ across the substrate; a plurality of Raman-active linker molecules directly bound to the metallic micro- or nanostructures; and a plurality of capture molecules directly bound to the Raman-active linker molecules. The disclosure also relates to systems, devices, and methods that use the substrates to determine the concentration of various analytes without a bound/free separation or a washing step/process.

The substrates, systems, devices, and methods provide, among other things, a significantly simpler, less costly, and highly sensitive platform for determining the concentration of analytes relative to conventional immunoassay clinical testing systems that require, among other things, a pair of antibodies (1st and 2nd reagents); competitive binding methods for small molecules, which can force a limitation in sensitivity; a bound/free separation or washing process, which can require costly magnetic particles as one reagent and can require costly and complicated hardware; and enzymatic reaction for chemiluminescence, which can require additional reaction time, thereby increasing the time necessary for analysis, and additional reagents, such as enzymes.

### DESCRIPTION OF THE DRAWINGS

Having thus described the presently disclosed subject matter in general terms, reference will now be made to the accompanying drawings, which are not necessarily drawn to scale, and wherein:

FIG. 1 is cartoon of a substrate according to the disclosure.

FIG. 2 is a scanning electron micrograph (SEM) of a sample substrate, such as the substrate shown in FIG. 1.

FIG. 3 is a scanning electron micrograph (SEM) of a sample substrate, such as the substrate shown in FIG. 1, the dark area is the hosting substrate such as quartz, silicon, et al.

FIG. 4 is a cartoon of a substrate, such as the substrate shown in FIG. 1.

FIG. 5 is a cartoon of plot of signal intensity as a function of shift of a Raman peak or feature when a plurality of “naked” Raman-active linker molecules directly bound to metallic micro- or nanostructures according to the disclosure.

FIG. 6 is a Raman spectrum obtained using the substrates described herein, wherein the top-most spectrum is that of a plurality of “naked” Raman-active linker molecules directly bound to the metallic micro- or nanostructures.

FIGS. 7A and 7B are spectra obtained using the substrates described herein, only the two figures focus on the area of the spectrum shown in FIG. 6 between 1565 cm⁻¹ and 1600 cm⁻¹ at concentrations ranging from 0 μLU/mL TSH to 7.5 μLU/mL TSH (FIG. 7A) and 0 μLU/mL TSH to 50 μLU/mL (FIG. 7B).

FIGS. 8A and 8B are plots of the Raman Peak Position in cm⁻¹ as a function of log of the analyte concentration corresponding to the Raman Shift data obtained as a function of analyte concentration shown in FIGS. 7A and 7B, respectively.

FIG. 9 is a cartoon of a device comprising a substrate, such as the one described in FIG. 1. In this example, the device can take the form of a 10 mm×10 mm chip comprising a plurality of regions on the substrate. The regions can be of any suitable size, as can the chip. But in this example, the device can have four regions, each region having a known concentration of an analyte pre-coated onto the substrate.

FIG. 10 is a cartoon of a “multiplexed” device, where a first region comprises a first Raman-active linker molecule and a first capture molecule; a second region comprises a second Raman-active linker molecule and a second capture molecule; a third region comprises a third Raman-active linker molecule and a third capture molecule; and a fourth region comprises a fourth Raman-active linker molecule and a fourth capture molecule.

FIGS. 11A-11D represent the results from curve fitting operations and “zero point” identification.

FIG. 12A is a plot of the Raman Peak Position in cm⁻¹ as a function of analyte concentration (T4, also known as DL-thyroxine).

FIGS. 12B and 12C are plots of the “true” concentration of T4 and how it correlates to the T4 concentration predicted using the methods described herein.

FIG. 13A is a plot of the Raman Peak Position in cm⁻¹ as a function of analyte concentration (testosterone).

FIGS. 13B and 13C are plots of the “true” concentration of testosterone and how it correlates to the testosterone concentration predicted using the methods described herein.

### DESCRIPTION

Reference will now be made in detail to certain embodiments of the disclosed subject matter. While the disclosed subject matter will be described in conjunction with the enumerated claims, it will be understood that the exemplified subject matter is not intended to limit the claims to the disclosed subject matter.

### Definitions

Specific binding partner: A member of a pair of molecules that interact by means of specific noncovalent interactions that depend on the three-dimensional structures of the molecules involved. Typical pairs of specific binding partners include antigen-antibody, hapten-antibody, hormone-receptor, nucleic acid strand-complementary nucleic acid strand, substrate-enzyme, inhibitor-enzyme, carbohydrate-lectin, biotin-avidin, and virus-cellular receptor.
Analyte: The term “analyte” includes both the actual molecule to be assayed and analogues and derivatives thereof when such analogues and derivatives bind another molecule used in the assay in a manner substantially equivalent to that of the analyte itself. The analyte is an example of a specific binding partner. Examples of analytes contemplated herein include, but are not limited to, analytes having a molecular weight of less than about 40 kDa, less than about 30 kDa, less than about 20 kDa, less than about 10 kDa, less than about 5 kDa, less than about 1 kDa, less than about 500 kDa, less than about 50 kDa, less than about 5 kDa, less than about 1 kDa, less than about 800 Da, less than about 500 Da, less than about 300 Da, less than about 200 Da, such as from about 200 Da to about 40 kDa, about 200 Da to about 1 kDa, about 200 Da to about 800 Da, about 750 Da to about 2 kDa or about 1 kDa to about 10 kDa. Examples of analytes contemplated herein, therefore include, but are not limited to, testosterone (288.42 Da), T4 (also known as DL-thyroxine; 776.87 Da), and thyroid stimulating hormone (TSH; approximately 28-30 kDa, which includes a 14 kDa alpha subunit and a 15 kDa beta subunit).
Antibody: The term “antibody” includes both intact antibody molecules of the appropriate specificity and antibody fragments (including Fab, F(ab′), F(ab′)2, and Fv fragments), as well as chemically modified intact molecules and antibody fragments, including hybrid molecules assembled by in vitro reassociation of subunits. Also included are genetically engineered antibodies of the appropriate specificity and/or affinity, including single-chain derivatives. Both polyclonal and monoclonal antibodies are included unless otherwise specified. Specific examples of antibodies contemplated herein antibodies having an affinity for a target analyte, such as prostate specific antigen (PSA), creatine kinase MB (CKMB) isoenzyme, cardiac troponin I (cTnI) protein, thyroid-stimulating hormone (TSH), influenza A (Flu A) antigen, influenza B (Flu B) antigen, and respiratory syncytial virus (RSV) antigen. Antibodies for such target analytes are known in the art.
Aptamer: The term “aptamer” includes nucleic acid molecules having specific binding affinity to molecules through interactions other than classic Watson-Crick base pairing. Aptamers, like peptides generated by phage display or monoclonal antibodies (“mAbs”), are capable of specifically binding to selected targets and modulating the target's activity, e.g., through binding aptamers may block their target's ability to function. Created by an in vitro selection process from pools of random sequence oligonucleotides, aptamers have been generated for over 100 proteins including growth factors, transcription factors, enzymes, immunoglobulins, and receptors. A typical aptamer is 10-15 kDa in size (30-45 nucleotides), binds its target with sub-nanomolar affinity, and discriminates against closely related targets (e.g., aptamers will typically not bind other proteins from the same gene family). A series of structural studies have shown that aptamers are capable of using the same types of binding interactions (e.g., hydrogen bonding, electrostatic complementarity, hydrophobic contacts, steric exclusion) that drive affinity and specificity in antibody-antigen complexes.
Sample: The term “sample” as used herein, refers to any fluid (e.g., a biological fluid from a subject) that can be applied to an assay device, directly or indirectly, and that contains or may contain an analyte, including, but not limited to, serum, plasma, whole blood, saliva, urine, cerebrospinal fluid, fecal extracts, material contained in a swab, such as a throat swab, or other fluids.
Geometrically anisotropic nanostructures/microstructures: The term “geometrically anisotropic” as used herein in conjunction with nano- and microstructures, refers to geometrically anisotropic structures that can exhibit anisotropic properties in the directions along and perpendicular to their long axes. Any and all geometrically anisotropic nanostructures/microstructures are contemplated herein, so long as they the presence of the anisotropy provides improved plasmonic properties because of the concentration of the E-field at a highest point (e.g., tips/summits) of the respective nanostructures/microstructures.

The term “about” as used herein can allow for a degree of variability in a value or range, for example, within 10%, within 5%, or within 1% of a stated value or of a stated limit of a range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more.

The term “substantially no” as used herein refers to less than about 30%, 25%, 20%, 15%, 10%, 5%, 3%, 2%, 1%, 0.5%, 0.1%, 0.05%, 0.001%, or at less than about 0.0005% or less or about 0% or 0%.

### Substrates

The instant disclosure generally relates to near-infrared dyes and their use as surface-enhanced Raman scattering (SERS) reporter molecules. For example, the disclosure relates to a substrate comprising:
a micro- or nanostructured periodic array comprised of a plurality of anisotropic (e.g., geometrically anisotropic) metallic micro- or nanostructures, wherein each of the plurality of nanostructures induce an average maximum and substantially uniform plasmonic field greater than 10⁸ (e.g., greater than about 10⁹, greater than about 10¹⁰, greater than about 10¹¹; about 10⁸ to about 10¹¹; about 10⁹ to about 10¹⁰; or about 10⁹ to about 10¹¹) optionally across substantially the entire substrate (for example, across at least a portion of the substrate and, in some instances, across substantially the entire substrate);
a plurality of Raman-active linker molecules directly bound to the metallic micro- or nanostructures; and
a plurality of capture molecules directly bound to the Raman-active inker molecules.

The substrate comprising the micro- or nanostructured periodic array comprised of a plurality of anisotropic (e.g., geometrically anisotropic) metallic micro- or nanostructures can be any suitable substrate. For example, the substrate can comprise at least one derivatizable metal that can be derivatized to form a covalent bond to the plurality of Raman-active linker molecules. The substrate can comprise, for example, at least one of gold and silver, and oxides of gold and silver.

Contemplated herein are substrates wherein at least a portion of the substrate comprises a M-I-M structure, wherein a top metallic layer comprises the micro- or nanostructured periodic array comprised of a plurality of anisotropic (e.g., geometrically anisotropic) metallic micro- or nanostructures. The insulating layer can be made of any suitable material, such as silicon nitride.

Also contemplated herein are substrates wherein at least a portion of the substrate comprises a nanoprism array or a silicon nanopillar array. See, e.g., ACS Appl. Nano Mater. 1: 5994-5999 (2018) describing plasmonic-diffractive hybrid sensors based on Au nanoprisms, which is incorporated by reference as if fully set forth herein; and ACS Nano 5: 4046-4055 (2011) describing periodic arrays of Au-capped, vertically aligned silicon nanopillars that are embedded in an Au plane upon a Si substrate, which is incorporated by reference as if fully set forth herein.

The substrates described herein can have a variety of features including roughness. For example, the substrate can have a roughness (e.g., a root-mean-square (RMS) roughness) of at least about 15 nm, at least about 30 nm, at least about 50 nm, at least about 100 nm, at least about 500 nm, at least about 750 nm, from about 1 nm to about 750 nm, about 10 nm to about 50 nm, about 1 nm to about 15 nm or about 5 nm to about 30 nm. The surface roughness can be determined using any suitable method including ISO 4287:1997, which is incorporated by reference as if fully set forth herein.

The plurality of anisotropic (e.g., geometrically anisotropic) metallic micro- or nanostructures can be made of any suitable metal, including at least one of gold and silver, and oxides of gold and silver. The micro- or nanostructures can have any suitable average height. For example, micro- or nanostructures can have an average height of from about 50 nm to about 5000 nm (e.g., from about 50 nm to about 500 nm, about 100 nm to about 1000 nm, about 500 nm to about 2500 nm, about 250 nm to about 2000 nm or about 75 nm to about 200 nm). The average height of the micro- or nanostructures can be determined using any suitable method including scanning electron microscopy.

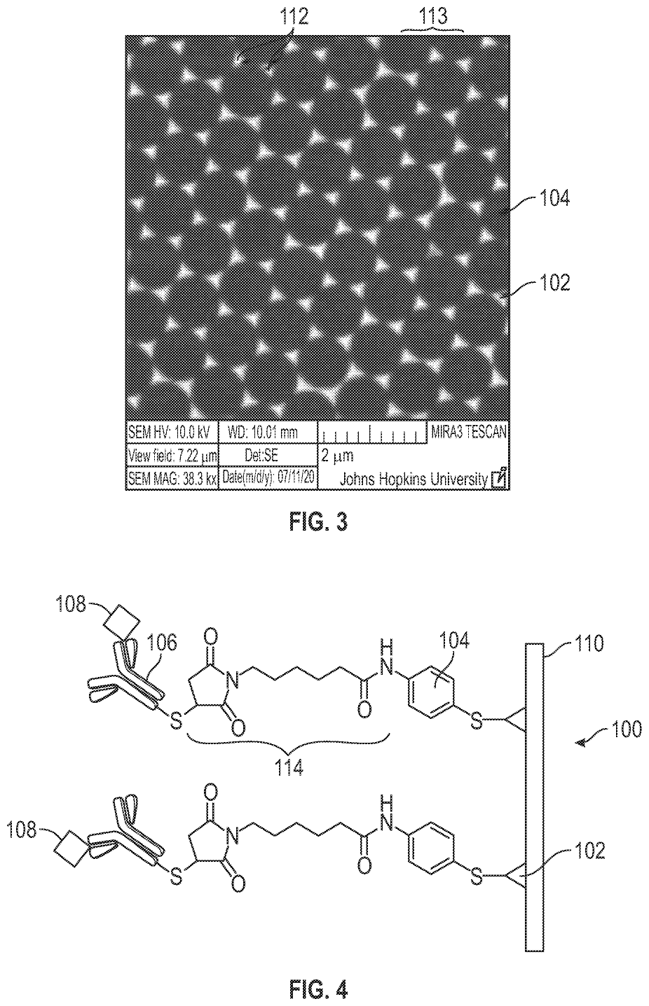

The plurality of anisotropic (e.g., geometrically anisotropic) metallic micro- or nanostructures can have any suitable periodicity. For example, plurality of anisotropic (e.g., geometrically anisotropic) metallic micro- or nanostructures can have a periodicity of from about 200 nm to about 5000 nm. (e.g., from about 250 nm to about 500 nm, about 200 nm to about 1000 nm, about 500 nm to about 2500 nm, about 250 nm to about 2000 nm or about 300 nm to about 800 nm). Making reference to FIG. 3, an example of “periodicity” is the center-to-center distance 113 between two closest polystyrene beads. The beads have been removed in FIG. 3 but have left behind an empty circular area uncovered by metal, gold in this case.

The plurality of anisotropic (e.g., geometrically anisotropic) metallic micro- or nanostructures can have any suitable shape. For example, the micro- or nanostructures are at least one of a geometric shape, a plurality of edges, or a plurality of steps. The geometric shapes are at least one of spheres, tetrahedrons, cubes, rods, cones, cylinders, triangular prisms, trigonal pyramids, square pyramids, or hexagonal pyramids. It should be understood, however, that the geometric shapes that the micro- or nanostructures can take, can approximate the aforementioned geometric shapes. In other words, the geometric shapes do not need to be exact (e.g., a perfect trigonal pyramid) but can have features that deviate from the exact geometric shapes.

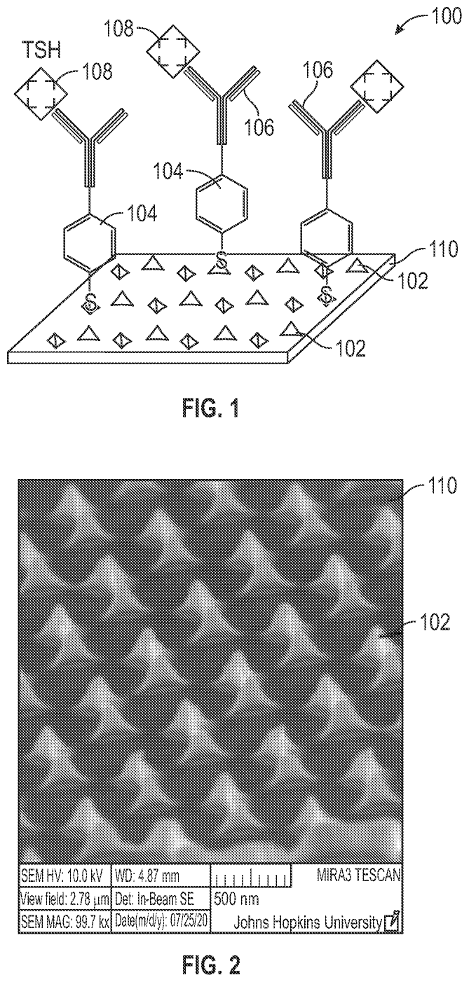

FIG. 1 is an example of a substrate 100 comprising plurality of anisotropic (e.g., geometrically anisotropic) metallic micro- or nanostructures 102, wherein the plurality of anisotropic (e.g., geometrically anisotropic) metallic micro- or nanostructures 102 are shown as trigonal pyramids. Bound to the plurality of anisotropic (e.g., geometrically anisotropic) metallic micro- or nanostructures 102 are a plurality of Raman-active linker molecules 104 directly bound to the metallic micro- or nanostructures 102; and a plurality of capture molecules 106 directly bound to the Raman-active linker molecules 104. In this particular example, the Raman-active linker molecules 104 are 4-aminothiophenol (ATP; amine group not shown); and the capture molecules 106 are antibodies having an affinity for an analyte 108, in this case thyroid-stimulating hormone (TSH).

The substrate can comprise a base layer or a plurality of base layers, each layer formed from the same material or from a different material. The base layers can comprise base layers that generate no Raman signal or a Raman signal that is substantially different from the spectral signature of the Raman-active linker molecules described herein. Examples of base layers include base layers comprising quartz, silica, silicon, glass, metal or a polymeric material (e.g., polydimethylsiloxane (PDMS) and polysilaxanes generally, polyethylene terephthalate (PTE), polyethylene (PE), and polypropylene (PP)). Once again making reference to FIG. 1, the substrate is numeral 110.

As mentioned herein, the geometric shapes do not need to be exact (e.g., a perfect trigonal pyramid) but can have features that deviate from the exact geometric shapes. Indeed, making reference to FIG. 2, one can see that the geometric shapes shown in the scanning electron micrograph (SEM) approximate the shape of a trigonal pyramid. But the top of the geometric shapes shown in FIG. 2 are slightly rounded.

Once again making reference to FIG. 2, in that example, the micro- or nanostructures are grouped to form a repeating pattern across at least a portion of the substrate. In this example, the repeating pattern is can be seen as a repeating hexagon pattern or a repeating pattern that follows that of a hexagonal lattice, where each of the plurality of anisotropic (e.g., geometrically anisotropic) metallic micro- or nanostructures 102 are located/formed on substrate 110. See FIG. 3, which is a top view SEM of the array shown in FIG. 2. The repeating pattern formed by the plurality of anisotropic (e.g., geometrically anisotropic) metallic micro- or nanostructures 102 can be any suitable repeating pattern. For example, the repeating pattern can be at least one of a triangular, a square, a hexagonal or a circular repeating pattern across at least a portion of the substrate. Making reference to FIG. 3, the repeating pattern formed by the plurality of anisotropic (e.g., geometrically anisotropic) metallic micro- or nanostructures 102 can be considered circular by virtue of the fact that edges 112 of the plurality of anisotropic (e.g., geometrically anisotropic) metallic micro- or nanostructures 102 (only two edges 112 are noted) form a circle.

### Capture Molecules

The capture molecules contemplated herein can be any suitable capture molecules with at least one of an appropriate affinity and an appropriate selectivity for its specific binding partner. The capture molecules contemplated herein form a specific binding pair with a binding partner. Examples of capture molecules include but are not limited to at least one of an antibody, an antibody fragment, a fusion protein, an aptamer, and an analyte. The capture molecules can have any suitable affinity for its specific binding partner. For example, the capture molecules can have an affinity for their specific binding partner, defined by an equilibrium dissociation constant (KD), of less than about 500 pM (e.g., less than about 250 pM, less than about 100 pM, less than about 50 pM, less than about 10 pM, less than about 5 pM, less than about 1 pM, less than about 500 fM, from about 500 fM to about 500 pM, about 500 pM to about 1 pM, about 1 fM to about 500 fM or about 750 fM to about 1 pM).

### Raman-Active Linker Molecules

The plurality of Raman-active linker molecules can be any suitable Raman-active linker molecules, including Raman-active chromophores. For example, the Raman-active linker molecules can comprise at least one of an organic or an organometallic molecule having a length along is largest axis of less than 40 nm. Examples of Raman-active linker molecules include Raman-active linker molecules with functional groups that make them amenable to conjugation with the capture molecules described herein (e.g., TSH MoAb) through any suitable chemistry (e.g., NHS/EDO chemistry). Examples of “functional groups” include at least one of carboxylic acids (—CO₂H), amines (e.g., —NH₂ and —NHR, wherein R can be alkyl or arylalkyl), and thiols (—SH). Examples of Raman-active linker molecules include but are not limited to molecules formed from 4-mercaptobenzoic acid, 4-aminothiophenol, 6-mercaptopurine, 8-aza-adenine, N-benzoyladenine, 2-mercapto-benzimidazole, 4-amino-pyrazole[3,4-d]pyrimidine, zeatin, methylene blue, 9-amino-acridine, ethidium bromide, Bismarck brown Y, N-benzyl-aminopurine, thionin acetate, 3,6-diaminoacridine, 6-cyanopurine, 4-amino-5-imidazole-carboxamidehydrochloride, 1,3-diiminoisoindoline, rhodamine 6G, crystal violet, basic fuchsin, aniline blue diammonium salt, N-[(3-(anilinomethylene)-2-chloro-1-cyclohexen-1-yl)methylene]anilinemonohydro-chloride, O-(7-azabenzotriazol-1-yl)-N,N,N′,N′-tetramethyluroniumhexafluoro-phosphate, 9-aminofluorene hydrochloride, basic blue, 1,8-diamino-4,5-dihydroxyanthraquinone, proflavine hemisulfate salt hydrate, 2-amino-1,1,3-propenetricarbonitrile, variamine blue RT salt, 4,5,6-triaminopyrimidine sulfate salt, 2-amino-benzothiazole, melamine, 3-(3-pyridylmethylamino)propionitrile, silver(I) sulfadiazine, acriflavine, 4-amino-6-mercaptopyrazole[3,4-d]pyrimidine, 2-minopurine, adenine thiol FAD fluoroadenine, 4-Amino-6-mercapyopyrazole[3,4-d]pyrimidine, rhodamine 110, adenine, 5-amino-2-mercaptobenzimidazole, acridine orange hydrochloride, cresyl violet acetate, acriflavine neutral, dimidium bromide, 5,10,15,20-tetrakis(N-methyl-4-pyridinio)porphyrin tetra(p-toluenesulfonate), 5,10,15,20-tetrakis(4-trimethylaminophenyl)porphyrin tetra(p-toluenesulfonate), 3,5-diaminoacridine hydrochloride, propidium iodide (3,8-diamino-5-(3-diethylaminopropyl)-6-phenylphenanthridinium iodidemethiodide), trans-4-[4-(dimethylamino)styryl]-1-methylpyridinium iodide, and 4-((4-(dimethylamino)phenyl)azo)benzoic acid, succinirnidyl ester or derivatives thereof.

The plurality of Raman-active linker molecules can be separated from the substrate by a divalent linker. In addition, or alternatively, the plurality of metallic micro- or nanostructures can be separated from the Raman-active linker molecules by a divalent linker. Examples of divalent linkers include organic linkers. Divalent linkers include but are not limited to at least one of an S(O)x group (wherein x is 0, 1 or 2), an alkyl, a carbonyl, a carboxylate, an amide, a polyoxyalkylene, a maleimide group and an amino acid radical of the formula —(O)C—(CR1R2)n-NH—, wherein R1 and R2 are each independently H, alkyl or an amino acid side chain, and n is an integer from 1 to 5. Examples of divalent linkers are linkers comprising groups of the formulae:

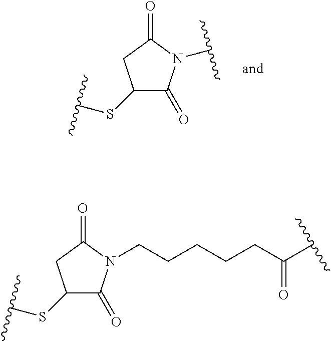

Making reference to FIG. 4, such a linker 114 can be connected on one terminus to, e.g., an 4-aminothiophenol Raman-active linker molecule 104 and to an antibody capture molecule at the other terminus. The Raman-active linker molecule 104 is, in turn, bound to substrate 110 comprising a plurality of metallic micro- or nanostructures 102.

### Devices and Systems

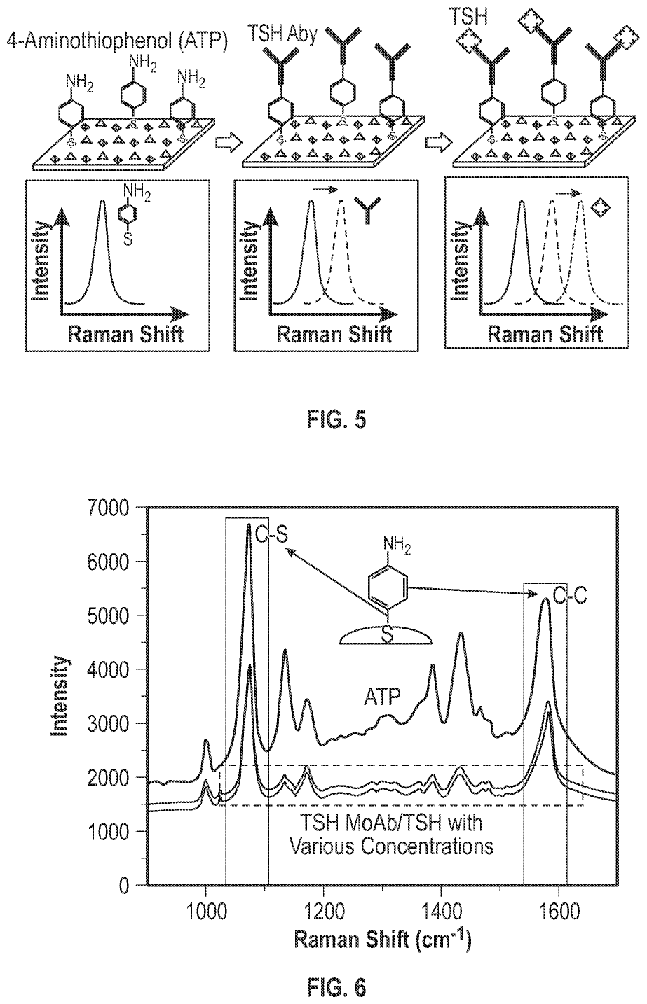

One of the features of the substrates described herein that makes them particularly applicable to devices and systems used for detecting analytes is that the Raman-active linker molecules located on the substrate exhibit a shift of a Raman peak or feature when the capture molecule binds an analyte. Making reference to FIG. 5, FIG. 5 depicts a cartoon of plot of signal intensity as a function of shift of a Raman peak or feature when a plurality of “naked” Raman-active linker molecules directly bound to the metallic micro- or nanostructures. In this example, the “naked” Raman-active linker molecules directly bound to the metallic micro- or nanostructures are molecules derived from 4-aminothiophenol. FIG. 5 also depicts a plot of signal intensity as a function of shift of a Raman peak or feature (e.g., in cm⁻¹) when a plurality of capture molecules are directly bound to the Raman-active linker molecules. In this example, the capture molecules are antibodies that bind TSH. Finally, FIG. 5 depicts a plot of signal intensity as a function of shift of a Raman peak or feature when a plurality of capture molecules is directly bound to their specific binding partner, namely, TSH, to form a specific binding pair. In the specific example given in FIG. 5, the Raman-active linker molecules exhibit a shift of a Raman peak or feature (e.g., a shoulder on a peak) in a higher wavenumber direction when a capture molecule binds an analyte. But situations are contemplated where the Raman-active linker molecules exhibit a shift of a Raman peak or feature in a lower wavenumber direction when a capture molecule binds an analyte. Regardless of whether the shift of a Raman peak or feature in a lower or higher wavenumber direction when a capture molecule binds an analyte, the shift of a Raman peak or feature is proportional to concentration of the analyte. Alternatively, regardless of whether the shift of a Raman peak or feature in a lower or higher wavenumber direction when a capture molecule binds an analyte, the Raman shift is inversely proportional to concentration of the analyte.

Making reference to FIG. 6, FIG. 6 shows an actual spectrum obtained using the substrates described herein, wherein the top-most spectrum is that of a plurality of “naked” Raman-active linker molecules directly bound to the metallic micro- or nanostructures. In this example, the “naked” Raman-active linker molecules directly bound to the metallic micro- or nanostructures are molecules derived from 4-aminothiophenol (ATP). FIG. 6 also depicts a plot of signal intensity as a function of Raman shift (e.g., in cm⁻¹) when a plurality of capture molecules bind to their specific binding partner, namely, TSH, to form a specific binding pair, at various ATP concentrations.

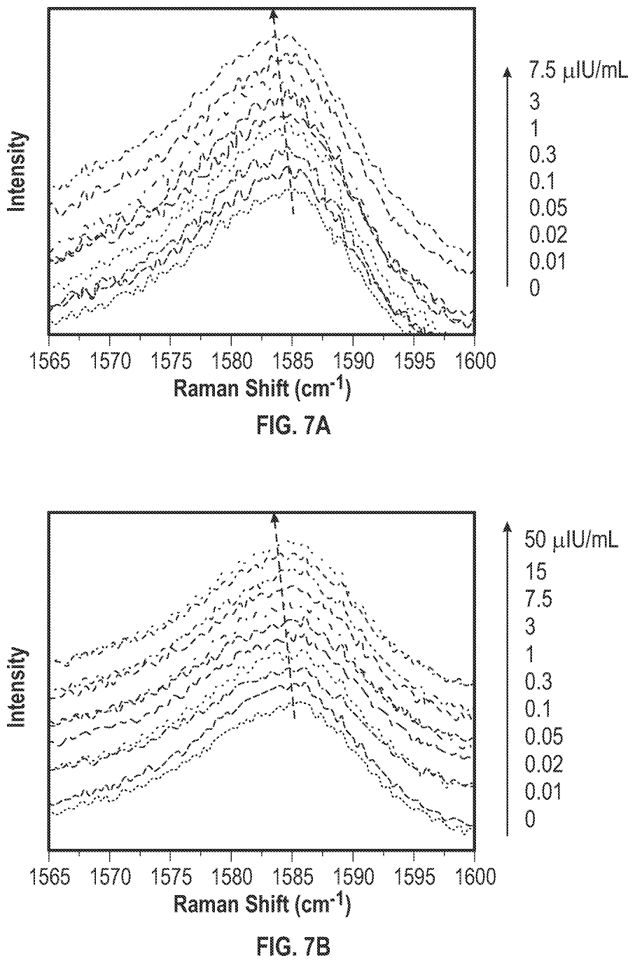

FIGS. 7A and 7B are spectra obtained using the substrates described herein, only the two figures focus on the area of the spectrum shown in FIG. 6 between 1565 cm⁻¹ and 1600 cm⁻¹ at concentrations ranging from 0 μLU/mL TSH to 7.5 μLU/mL TSH (FIG. 7A) and 0 μLU/mL TSH to 50 μLU/mL (FIG. 7B). In both of these examples, the Raman-active linker molecules exhibit a shift of a Raman peak or feature in a lower frequency direction when a capture molecule binds an analyte. The dashed arrow in FIGS. 7A and 7B is angled toward the left (i.e., toward a lower frequency direction), thus showing that at the highest TSH concentration (top most spectrum), the peak/feature at approximately 1585 cm⁻¹ has shifted to the left relative to the spectrum obtained in the absence of TSH (i.e., 0 μLU/mL TSH; bottom most spectrum).

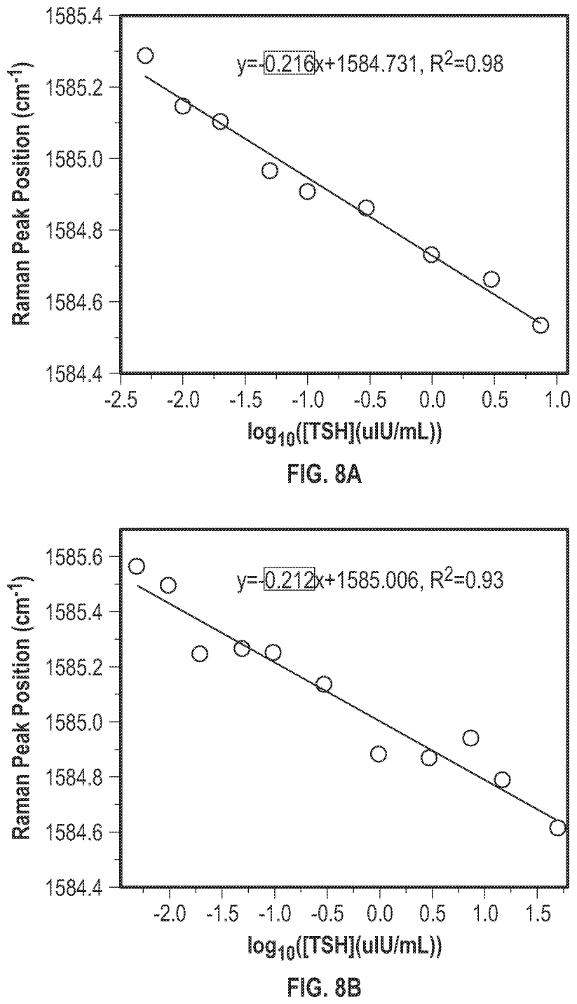

FIGS. 8A and 8B are plots of the Raman Peak Position in cm⁻¹ as a function of log₁₀ of the analyte concentration corresponding to the Raman Shift data obtained as a function of analyte concentration shown in FIGS. 7A and 7B, respectively. The plots, and corresponding linear interpolations, demonstrate that the Raman Peak Position in cm⁻¹ shifts linearly as a function of log₁₀ of the analyte concentration, over the analyte concentrations tested.

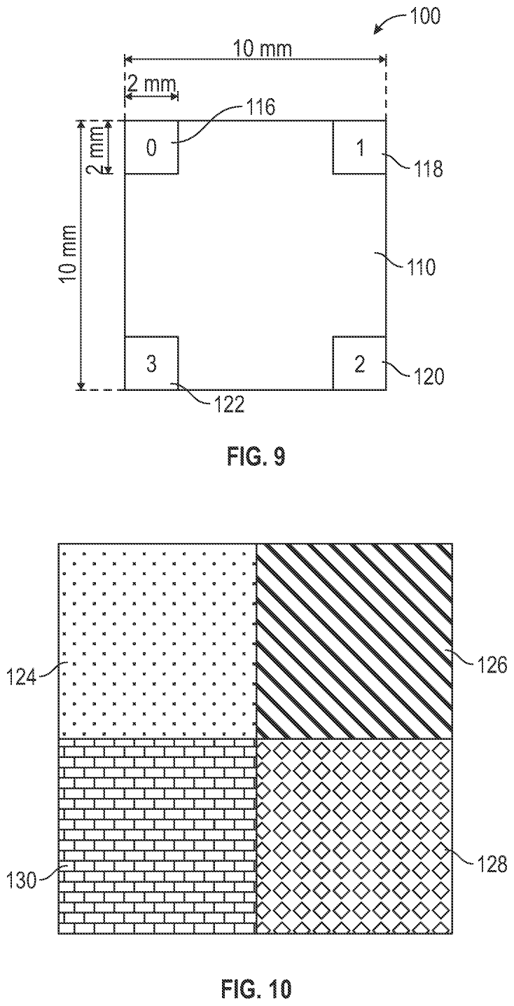

Making reference to FIG. 1, the experimental results depicted in FIGS. 7A, 7B, 8A, and 8B were collected using a device comprising substrate 100 comprising a plurality of anisotropic (e.g., geometrically anisotropic) metallic micro- or nanostructures 102, wherein the plurality of anisotropic (e.g., geometrically anisotropic) metallic micro- or nanostructures 102 took the form of trigonal pyramids, such as those shown in FIG. 2. As shown in FIG. 1, bound to the plurality of anisotropic (e.g., geometrically anisotropic) metallic micro- or nanostructures 102 are a plurality of Raman-active linker molecules 104 directly bound to the metallic micro- or nanostructures 102; and a plurality of capture molecules 106 directly bound to the Raman-active linker molecules 104. In this particular example, the Raman-active linker molecules 104 are 4-aminothiophenol (ATP; amine group not shown); and the capture molecules 106 are antibodies having an affinity for an analyte 108, in this case thyroid-stimulating hormone (TSH). Envisioned herein are devices, such as the one shown in FIG. 9, comprising a substrate, such as the one described in FIG. 1. In this example, the device can take the form of a 10 mm×10 mm chip (also referred to herein as a “test chip”) comprising a plurality of regions on the substrate. The regions can be of any suitable size, as can the chip. But in this example, the device can have four regions, each region having a known concentration of an analyte pre-coated onto the substrate. Thus, for example, region 116 can comprise no analyte, whereas regions 118, 120, and 122 can comprise low, intermediate, and high concentrations of the analyte. Such a device would permit having an on-board calibration for the chip. In sum, contemplated herein is a substrate or device comprising at least one region (e.g., at least two regions, at least three regions, at least four regions, at least five regions or more) comprising no analyte; at least one other region comprising a known concentration of an analyte; and at least one region comprising a sample with an unknown concentration of an analyte.

Other devices are also contemplated herein where, in addition to the on-board calibration feature or alternatively to that feature, the device can comprise a plurality of regions, each of the plurality of regions having two, three, four, or more different combinations of at least one of Raman-active linker molecules and capture molecules.

Making reference to FIG. 10, as an example of such a “multiplexed” device, where a first region 124 comprises a first Raman-active linker molecule and a first capture molecule; a second region 126 comprises a second Raman-active linker molecule and a second capture molecule; a third region 128 comprises a third Raman-active linker molecule and a third capture molecule; and a fourth region 130 comprises a fourth Raman-active linker molecule and a fourth capture molecule. In this example, the first, second, third, and fourth Raman-active linker can be the same or different. In addition to, or alternatively, the first and second capture molecules can be the same or different; and the third and fourth capture molecules can be the same or different, so long as there are at least two regions, at least three regions or at least four regions (or more) having different capture molecules. In another example, the substrate or device comprises a first region comprising a first Raman-active linker molecule and a first capture molecule; and a second region comprising a second Raman-active linker molecule and a second capture molecule, wherein the first and second Raman-active linkers are the same or different; and the first and second capture molecules are the same or different. In yet another example, the substrate or device comprises a first region comprising a first Raman-active linker molecule and a first capture molecule; and a second region comprising a second Raman-active linker molecule and a second capture molecule, wherein the first and second Raman-active linkers are the same; and the first and second capture molecules are different.

In examples where the “multiplexed” device comprises a plurality of regions, such as at least two regions comprising two different capture molecules, such that one can measure the concentration of at least two different analytes. In examples where the “multiplexed” device comprises at least three regions comprising three different capture molecules, one can measure the concentration of at least three different analytes. And where the “multiplexed” device comprises at least four regions comprising four different capture molecules, one can measure the concentration of at least four different analytes. FIG. 10 is a depiction of a “multiplexed” device comprising four different regions comprising four different capture molecules such that one can measure the concentration of four different analytes. The multiplexed devices contemplated herein can be used, for example, to measure/evaluate a subject's prostate health index by measuring the concentration of PSA, FreePSA, and p2PSA; to conduct a thyroid panel by measuring the concentration of free thyroxine, free triiodothyronine, thyroid stimulating hormone, thyroglobulin antibodies, and thyroid peroxidase antibodies; or to conduct a cardiac panel where what is measured is cardiac proteins creatine-kinase-MB (CK-MB), myoglobin, and troponin I (cTnI) in whole blood and plasma specimens.

The substrates and devices (e.g., a chip, such as a multiplexed chip or a microfluidic device) described herein can be used in a system for quantifying a biomarker in a sample. The system can comprise, among other things, the substrates or devices described herein; a light source; a signal detector; and a computational device. The signal detector detects a shift of a Raman peak or feature in a Raman spectrum of the plurality of Raman-active linker molecules; and the computation device uses Raman mapping to measure the Raman spectral peak wavelength of the Raman-active linker molecules. As described herein, the biomarker can be located in (for example, can be in a solution comprising) a biological fluid, such as at least one of whole blood, plasma, serum, saliva, and urine.

The light source can be any suitable light source. For example, the light source can be broad spectrum light or a monochromatic light source having a wavelength that matches the wavelength of at least one Raman-active linker molecule on the substrate. Suitable light sources include light from a laser, such as a continuous wave laser. In other examples, the source can be from a solid state UV laser. Thus, for example, the light source can be from at least one of argon lasers, krypton, helium-neon, helium-cadmium types, and diode lasers. Or the light source can be from one or more continuous wave lasers, arc lamps, or LEDs.

For example, the systems contemplated herein can comprise multiple (one or more) light sources. When the system comprises multiple light sources, each of the light sources can emit electromagnetic radiation at the same wavelengths. Alternatively, each of the light sources can emit light of different wavelengths in order to accommodate the different absorption spectra of different Raman-active linker molecules present in a device.

Specific examples of light sources include a Triton UV laser (diode-pumped Q-switched Nd:YLF laser, Spectra-Physics) operating at a wavelength of 349 nm, a focused beam diameter of 5 μm, and a pulse duration of 20 ns; an X-cite 120 illumination system (EXFO Photonic Solutions Inc.) with a XF410 QMAX FITC and a XF406 QMAX red filter set (Omega Optical); and a diode laser is a Oclaro HL63133DG laser with a peak power of 170 mW operating at a wavelength of 635 nm. In another example embodiment, the diode laser is an Osram PL450B laser operating at 450 nm. Also included herein are integrated internal laser sources, integrated into Raman spectrophotometers with any suitable wavelengths (e.g., 785 nm, 638 nm, and 532 nm).

The light source can scan the surface (e.g., a portion or the entire surface) of the substrate or the device by, e.g., moving the light source, moving an x-y state on which the substrate or device is mounted, or a combination of moving the light source and the substrate or device. For example, one can designate a 100×100-point area to be scanned by the light source. The data obtained from the 100×100-point are to be scanned by the light source can then be assumed to be representative of the entire substrate or device.

The systems contemplated herein also include a signal detector that receives electromagnetic (EM) radiation from the Raman-active linker molecules located on the substrates described herein upon excitation using the light sources described herein. The signal detector can identify at least one cavity (e.g., a microcavity) emitting electromagnetic radiation from one or more labels. The signal detector can be any suitable signal detector, including a single wavelength signal detector; a multiple wavelength signal detector (e.g., a photodiode array signal detector).

Those of skill in the art will recognize that the systems described herein can also comprise various optical elements that can aid the collection of signals from the Raman-active linker molecules described herein. For example, the systems described herein can comprise appropriate lenses, optical filters, and gratings.

As discussed herein, the signal detector detects a shift of a Raman peak or feature in a Raman spectrum of the plurality of Raman-active linker molecules; and the computation device uses Raman mapping to measure the Raman spectral peak wavelength of the Raman-active linker molecules. Any suitable method for Raman mapping to measure the Raman spectral peak wavelength of the Raman-active linker molecules can be used, including methods described in J. Biophotonics 5: 220-229 (2012) (describing a method for acquiring high-spatial-resolution spectral maps, in particular for Raman micro-spectroscopy (RMS), by selectively sampling the spatial features of interest and interpolating the results); Analyst 137: 4119-4122 (2012) (describing a selective scanning method was used to measure spatially resolved Raman spectra of live Neospora caninum tachyzoites colonizing human brain microvascular-endothelial cells. The technique allowed the detection of nucleic acids, lipids and proteins linked to the parasites and their cellular micro-environment at ˜10× shorter acquisition time compared to raster scanning); Curr. Op. in Chem. Biol. 33: 16-24 (2016) (describing Raman microscopy with parallel spectral acquisition provides about two orders of magnitude improvement in imaging speed); Analytical Chemistry 90: 4461-4469 (2018) (describing reducing the total number of data points required for image generation in Raman microscopy by using sparse sampling strategies, in which the preceding set of measurements informed the next most information-rich sampling location); J. Biophotonics 13: e201960109 (2020) (describing a superpixel acquisition approach that can expedite acquisition between ˜×100 and ×10,000, as compared to point-by-point scanning by trading off spatial resolution); and Biosensors and Bioelectronics: 112863 (2020) (describing coarse Raman microscopy that is capable of rapidly mapping a sufficient number of cells for training a random forest classifier that can accurately predict the metastatic potential of cells at a single-cell level), all of which are incorporated by reference as if fully set forth herein.

Raman maps include spectra recorded at discrete points on the substrates and devices described herein. The Raman maps show the variation of any fitted parameter (e.g., intensity, width or position of one band) as a function of the point of analysis. For example, the Raman maps show the peak intensity, or the peak position, or the relative amount of peak shift with respect to a sample tested under zero analyte concentration. One general aim of smart mapping is to selectively sample spatial points on a substrate and interpolate the results. This can enable a significant reduction (˜10×-100×) reduction in the sampling time compared to raster-scanning without compromising on spectral signal-to-noise ratios and hence providing the same diagnostic performance. Raman mapping is useful at least in reducing point-to-point variability and offering a robust concentration estimation.

The computational device (e.g., a computer) can use any suitable algorithm (e.g., artificial intelligence and machine learning) to correlate at least one of spectral shift and spectral profile (e.g., spectral shift and intensity) to the concentration of an analyte. Alternatively, or in addition, the computational device can provide an estimate of the concentration of an analyte by analyzing patterns that occur together (e.g., frequent pattern expression). For example, the computational device can use principal component regression, partial least squares regression or support vector regression. Such algorithms can utilize a full multi-channel nature of spectral data instead of focusing on a few specific peaks, as there can often be latent but useful information in other spectral patterns that do not appear as significant to gross visual inspection. The use of full spectra in conjunction with the chemometric model (e.g., created from training samples) for prediction of concentration or a similar quantity (e.g., protein particle aggregation) in unknown samples is described at Advanced Healthcare Materials: 2001110 (2020) (describing facilitating rapid, point-of-care measurements, using a label-free surface-enhanced Raman spectroscopy (SERB) sensing platform that leverages the specificity of molecular vibrations and signal amplification on silver-coated silicon nanowires (Ag/SiNWs) for highly sensitive and reproducible quantification of glycated albumin as a robust biomarker for screening and monitoring of diabetes); and Analytica Chimica Acta 1081: 138-145 (2019) (describing the use of label-free Raman spectroscopy in conjunction with multivariate analysis), both of which are incorporated by reference as if fully set forth herein.

Also contemplated herein is a method of measuring a concentration of an analyte, the method comprising:
combining a sample with an unknown concentration of the analyte with the substrates or devices described herein;
impinging a light source on at least a portion of the substrate;
measuring a Raman-signal from the plurality of Raman-active linker molecules via a detector; and
utilizing Raman mapping to measure the concentration of the analyte.

The method can further comprise an incubation step, wherein a sample with an unknown concentration of the analyte is incubated with the substrates or devices described herein before the impinging and measuring steps. Those of skill in the art will know or will be able to determine a suitable incubation period. For example, the incubation period can be a three, five, seven, ten, 15, 20, 30, 40, 50 or 60 (or more) minute period. Alternatively or in addition, a rate method can be used to estimate the concentration of an analyte using the substrates, devices, and methods described herein, wherein one can, (e.g., combine a sample with an unknown concentration of the analyte with the substrates or devices described herein, and at some predetermined point (e.g., three minutes from the combining) measure a Raman-signal for a predetermined amount of time (e.g., ten seconds) to estimate the concentration of the analyte.

The methods described herein also include a method for detecting TSH concentration in a biological sample, the method comprising: locating a biological sample volume of at least about 50 μL or less on a chip; incubating the biological sample on the chip at 37±3° C. for at least about 15 minutes or less; and generating a report on the TSH concentration in the biological sample in about 20 minutes or less; the method having a measurable range of about 0.01 μLU/mL to about 50 μLU/mL; and the chip comprising: a plurality of gold nanostructures formed on a disposable test chip, 4-ATP as SERS active molecules bonded to a surface of the plurality of gold nanostructures through the thiol group; and TSH antibodies bonded (e.g., covalently) to the 4-ATP via the amine group.

One of ordinary skill in the art will recognize that the methods of the current disclosure can be achieved by administration of a composition described herein comprising at least one bronchodilator and at least one pulmonary surfactant via devices not described herein.

Values expressed in a range format should be interpreted in a flexible manner to include not only the numerical values explicitly recited as the limits of the range, but also to include all the individual numerical values or sub-ranges encompassed within that range as if each numerical value and sub-range were explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. In addition, it is to be understood that the phraseology or terminology employed herein, and not otherwise defined, is for the purpose of description only and not of limitation. Any use of section headings is intended to aid reading of the document and is not to be interpreted as limiting. Further, information that is relevant to a section heading can occur within or outside of that particular section. Furthermore, all publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

In the methods described herein, the steps can be carried out in any order without departing from the principles of the invention, except when a temporal or operational sequence is explicitly recited. Furthermore, specified steps can be carried out concurrently unless explicit claim language recites that they be carried out separately. For example, a claimed step of doing X and a claimed step of doing Y can be conducted simultaneously within a single operation, and the resulting process will fall within the literal scope of the claimed process.

Those skilled in the art will appreciate that many modifications to the embodiments described herein are possible without departing from the spirit and scope of the present disclosure. Thus, the description is not intended and should not be construed to be limited to the examples given but should be granted the full breadth of protection afforded by the appended claims and equivalents thereto. In addition, it is possible to use some of the features of the present disclosure without the corresponding use of other features. Accordingly, the foregoing description of or illustrative embodiments is provided for the purpose of illustrating the principles of the present disclosure and not in limitation thereof and can include modification thereto and permutations thereof.

### EXAMPLES

The disclosure can be better understood by reference to the following examples which are offered by way of illustration. The disclosure is not limited to the examples given herein.

### Example 1: Substrate Fabrication

Experimental conditions, including reagents, solvents, and processing parameters, for generating nanopyramids on an underlayer. The underlayer (quartz slides) is treated by immersion into an acidiperoxide solution solution (H₂SO₄:H₂O₂=3:1) under heating at 90° C. for 2 hours. The underlayer is then cleaned by ultrasonication for 10 minutes in acetone, ethanol, and DI water successively. Polystyrene beads are subsequently dip-coated onto the underlayer. Chromium and gold are deposited using e-beam evaporation. The deposition rates are 0.05 nm/s and 0.25 nm/s for chromium and gold deposition, respectively. The polystyrene beads are removed by ultrasonication for 10 minutes in ethanol. Gold nanopyramid arrays are obtained on the underlayer upon removal of the beads.

### Example 2: Protocol for SERS Detection of TSH on Gold Nanopyramid Arrays

Raman molecule functionalization: a substrate prepared as described in Example 1 is incubated in an ethanol solution of 10 mM Raman molecules (4-aminothiophenol). After 20 min, the substrate is washed using ethanol to remove excess reagents and then dried using compressed air.
TSH MoAb activation and functionalization: 10 μL of 20 μg/mL thyroid stimulating hormone (TSH; e.g., available from Thermo Fisher) monoclonal antibody (MoAb) in phosphate-buffered saline (PBS) is first mixed with 50 μL of PBS solution containing 50 mM sulfo-N-hydroxy succinimide (NHS; e.g., available from Sigma-Aldrich) and 200 mM EDC for 5 min. The 150 μL mixture is then dropped to cover the entire chip for incubation of 12 hours at room temperature. The chip is then washed using ACCESS™ wash buffer II (Beckman Coulter; 19.6 mM Tris, 150 mM NaCl, 0.1% NaN3, 0.1% ProClin 300, pH 8.31±0.05 at 25° C.) to remove excess reagents and dried using compressed air.
Bovine serum albumin (BSA) blocker: The substrate is further incubated in 10 mg/mL BSA in PBS buffer for 12 hours. The substrate is then washed using the ACCESS™ Wash Buffer II to remove excess reagents and dried using compressed air.
TSH Ag detection: The TSH MoAb-functionalized substrates are incubated into 50 μL of TSH Ag (varying concentrations). After 15 minutes reaction at 37° C., the substrate is placed under the Raman microscope for SERS spectral collection without any washing step. Alternatively, the substrate can be first washed using Access Wash Buffer and dried using compressed air before being placed under the Raman microscope for SERS spectral collection.

### Example 3: Signal Processing and Raman Shift Analysis

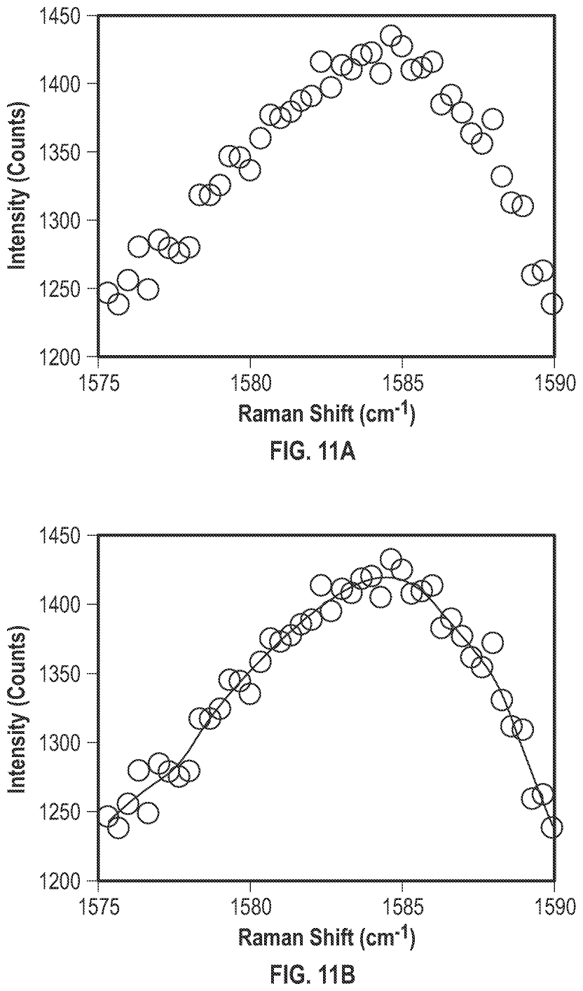

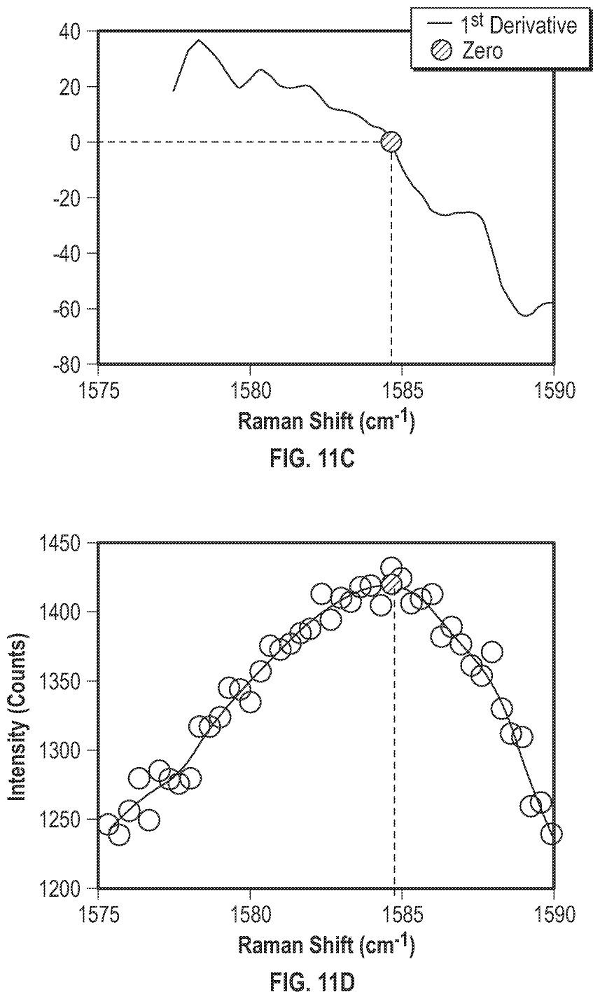

A “raw” Raman spectrum was obtained, an example of a portion of which is shown in FIG. 11A. A suitable curve smoothing operation was then performed on the raw Raman spectrum portion shown in FIG. 11A to obtain FIG. 11B. In this example, the curve smoothing operation is a cubic smoothing spline interpolation. The “zero point” is identified in the first derivative of the curve shown in FIG. 11B to obtain the curve in FIG. 11C. In this example, the “zero point” occurs at 1585 cm⁻¹. The “zero point” can then be superimposed on FIG. 11B (see FIG. 11D) and peak shifts are measured from the “zero point.”

### Example 4: SERS Detection of TSH on Gold Nanopyramid Arrays

This experiment was conducted in a similar fashion as described in Example 2, except that the TSH MoAb-functionalized substrates were incubated in 50 μL of TSH Ag (varying concentrations) for 20 minutes at 37° C. The substrate was placed under the Raman microscope for SERS spectral collection without any washing step. The acquisition time was 3 seconds with a 7× accumulation. The data for this experiment is shown in FIGS. 7-8, with FIG. 7 showing the peak shifts and FIG. 8 showing the correlation between peak shifts and TSH concentration determined from two different chips/substrates prepared according to the methods described herein.

### Example 5: SERS Detection of T4 on Gold Nanopyramid Arrays

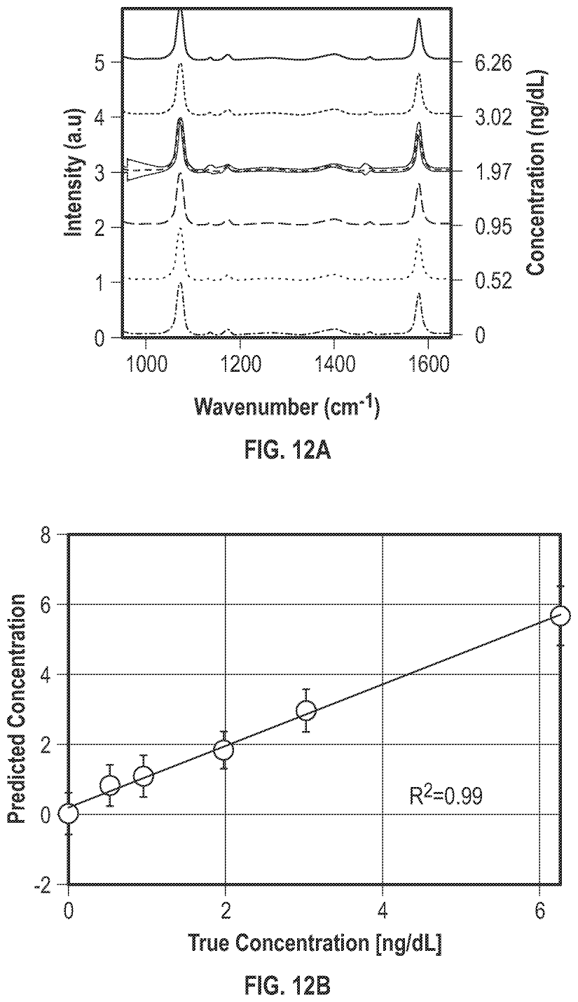

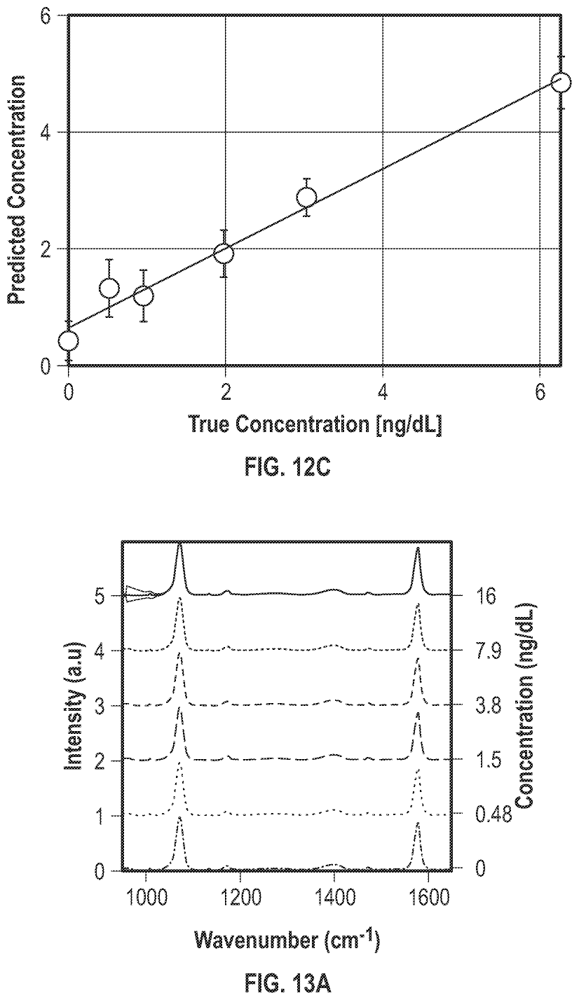

This experiment was conducted in a similar fashion as described in Example 2, except that the T4 MoAb-functionalized substrates were incubated in 50 μL of T4 Ag (varying concentrations) for 20 minutes at 37° C. The substrate was placed under the Raman microscope for SERS spectral collection. The acquisition time was 3 seconds with a 7× accumulation. The data for this experiment is shown in FIGS. 12A-12C, with FIG. 12A showing the SERS spectra and FIGS. 12B-12C showing the correlation between the predicted and “true” concentration by analyzing the spectral variations. These results also show that the methods described herein are sensitive and accurate enough to determine the concentration of analytes having a molecular weight as low as about the molecular weight of T4 (776.87 Da).

### Example 6 SERS Detection of Testosterone on Gold Nanopyramid Arrays

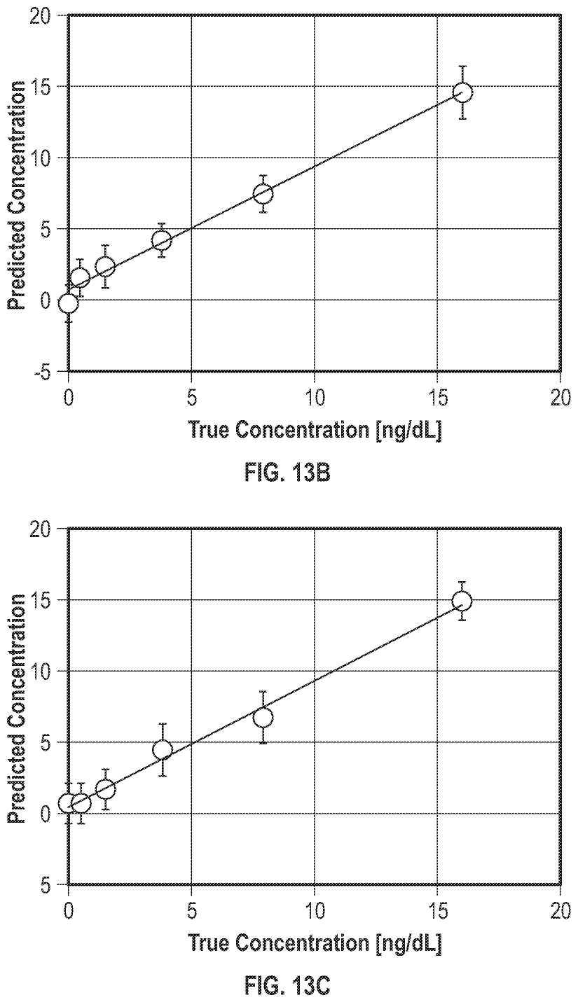

This experiment was conducted in a similar fashion as described in Example 2, except that the testosterone MoAb-functionalized substrates were incubated in 50 μL of testosterone Ag (varying concentrations) for 20 minutes at 37° C. The substrate was placed under the Raman microscope for SERS spectral collection. The acquisition time was 3 seconds with a 7× accumulation. The data for this experiment is shown in FIGS. 13A-13C, with FIG. 13A showing the SERS spectra and FIGS. 13B-13C showing the correlation between the predicted and “true” concentration by analyzing the spectral variations. These results also show that the methods described herein are sensitive and accurate enough to determine the concentration of analytes having a molecular weight as low as about the molecular weight of testosterone (288.42 Da).

1. A substrate comprising
a. a micro- or nanostructured periodic array comprised of a plurality of anisotropic metallic micro- or nanostructures, wherein each of the plurality of nanostructures induce an average maximum and substantially uniform plasmonic field greater than 10⁸ optionally across substantially the entire substrate;
b. a plurality of Raman-active linker molecules directly bound to the metallic micro- or nanostructures; and
c. a plurality of capture molecules directly bound to the Raman-active linker molecules,
wherein the shift of a Raman peak or feature is proportional or inversely proportional to concentration of the analyte.

2. The substrate of claim 1, wherein the capture molecules are at least one of an antibody, an antibody fragment, a fusion protein, an aptamer, and an analyte.

3. The substrate of claim 2, wherein the antibody, antibody fragment, fusion protein or aptamer has a Kd of at least 1 pM.

4. The substrate of claim 1, wherein the substrate is formed on a base layer.

5. (canceled)

6. The substrate of claim 4, wherein the base layer comprises quartz, silica, glass, metal or a polymeric material.

7. (canceled)

8. The substrate of claim 1, wherein the micro- or nanostructures have an average height of from about 50 to 5000 nm.

9. The substrate of claim 1, wherein the micro- or nanostructures have a periodicity of from about 200 nm to about 5000 nm.

10. The substrate of claim 1, wherein the micro- or nanostructures are at least one of a geometric shape, a plurality of edges, or a plurality of steps.

11. The substrate of claim 10, wherein the geometric shapes are at least one of trigonal pyramids, square pyramids, or hexagonal pyramids.

12.-15. (canceled)

16. The substrate of claim 1, wherein the plurality of Raman-active linker molecules comprise at least one of an organic or an organometallic molecule having a length along is largest axis of less than 40 nm.

17. (canceled)

18. The substrate of claim 1, wherein the Raman-active linker molecules exhibit a shift of a Raman peak or feature in a higher wavenumber direction when a capture molecule binds an analyte.

19. The substrate of claim 1, wherein the Raman-active linker molecules exhibit a shift of a Raman peak or feature in a lower wavenumber direction when a capture molecule binds an analyte.

20. (canceled)

21. (canceled)

22. The substrate of claim 1, wherein the plurality of Raman-active linker molecules comprise a Raman-active chromophore.

23. The substrate of claim 1, wherein at least a portion of the substrate comprises a metal-insulator-metal structure, a nanoprism array or a silicon nanopillar array.

24. The substrate of claim 1, wherein the plurality of Raman-active linker molecules are separated from the substrate by a divalent linker.

25. (canceled)

26. The substrate of claim 24, wherein each divalent linker is an organic linker.

27. The substrate of claim 26, wherein each divalent linker is at least one of a carboxylate, amide, polyoxyalkylene, maleimide group and an amino acid radical of the formula —(O)C—(CR¹R²)ₙ—NH—, wherein R¹ and R² are each independently H, alkyl or an amino acid side chain, and n is an integer front 1 to 5.

28.-29. (canceled)

30. A system for quantifying a biomarker in a sample, the system comprising:
a. the substrate of claim 1;
b. a light source;
c. a signal detector; and
d. a computational device;
e. wherein:
i. the signal detector detects a shift of a Raman peak or feature in a Raman spectrum of the plurality of Raman-active linker molecules; and
ii. the computation device uses Raman mapping to measure the Raman spectral peak wavelength of the Raman-active linker molecules.

31.-42. (canceled)

43. A method of measuring a concentration of an analyte, the method comprising:
a. combining a sample with an unknown concentration of the analyte with the substrate of claim 1;
b. impinging a light source on at least a portion of the substrate or device;
c. measuring a Raman-signal from the plurality of Raman-active linker molecules via a detector; and
d. determining the concentration of the analyte,
wherein the determining of the concentration of the concentration of the analyte comprises utilizing Raman mapping.

44.-48. (canceled)

49. A method for detecting TSH concentration in a biological sample, the method comprising:
locating a biological sample volume of at least about 50 μL or less on a chip; incubating the biological sample on the chip at 37±3° C. for at least about 15 minutes or less; and
generating a report on the TSH concentration in the biological sample in about 20 minutes or less;
the method having a measurable range of about 0.01 μLU/mL to about 50 μLU/mL; and the chip comprising:
a plurality of gold nanostructures formed on a disposable test chip,
4-ATP as SERS active molecules bonded to a surface of the plurality of gold nanostructures; and
TSH antibodies bonded to the 4-ATP.
